(19) United States
(12) Patent Application Publication
(10) Pub. No.: US 2024/0216084 A1
(43) Pub. Date: Jul. 4, 2024

(54) BRAIDED ARTICULATION ASSEMBLY FOR ELONGATE MEMBER

(71) Applicant: Auris Health, Inc., Redwood City, CA (US)

(72) Inventors: Sean N. Finson, Santa Clara, CA (US); Taylor E. Furtado, Campbell, CA (US); James Hayes, La Honda, CA (US)

(21) Appl. No.: 18/536,607

(22) Filed: Dec. 12, 2023

Related U.S. Application Data
(60) Provisional application No. 63/436,191, filed on Dec. 30, 2022.

(51) Int. Cl. A61B 34/30

(52) U.S. Cl. CPC A61B 34/30; A61B 2034/301

(57) ABSTRACT

An apparatus includes an elongate body, first and second tendons, and first and second stiffening members. The tendons extend through the elongate body and have respective distal ends that are fixedly secured relative to the distal end of the elongate body. The tendons are operable to drive articulation of the distal portion of the elongate body. The stiffening members have respective distal ends that are fixedly secured relative to the distal end of the elongate body. The first tendon and the first stiffening member are positioned adjacent to each other along the proximal portion; and are angularly offset from each other along the distal portion. The second tendon and the second stiffening member are positioned adjacent to each other along the proximal portion; and are angularly offset from each other along the distal portion.

### PRIORITY

This application claims the benefit of U.S. Pat. App. No. 63/436,191, entitled “Braided Articulation Assembly for Elongate Member,” filed Dec. 30, 2022, the disclosure of which is incorporated by reference herein.

### BACKGROUND

A variety of surgical instruments include an end effector for use in medical treatments and procedures conducted by a medical professional operator, including applications in robotically assisted surgeries. In the case of robotically assisted surgery, the surgeon may operate a master controller to remotely control the motion of such surgical instruments at a surgical site. The controller may be separated from the patient by a significant distance (e.g., across the operating room, in a different room, or in a completely different building than the patient); or quite near the patient in the operating room. The controller may include one or more hand input devices (e.g., joysticks, exoskeletal gloves, master manipulators, etc.), which are coupled by a servo mechanism to the surgical instrument. In one example, a servo motor moves a manipulator supporting the surgical instrument based on the surgeon's manipulation of the hand input devices. During the surgery, the surgeon may employ, via a robotic surgical system, a variety of surgical instruments including an ultrasonic blade, a surgical stapler, a tissue grasper, a needle driver, an electrosurgical cautery probes, etc. Each of these structures performs functions for the surgeon, for example, cutting tissue, coagulating tissue, manipulating a needle, grasping a blood vessel, dissecting tissue, or cauterizing tissue. A robotically-controlled instrument may be introduced into the patient via an incision, via a naturally occurring orifice, or otherwise.

While several robotic surgical systems and associated components have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a top plan view of an example of a robotic surgical system being used in a urological procedure.

FIG. 2 depicts a schematic view of different components of the robotic surgical system of FIG. 1.

FIG. 3 depicts enlarged views of other components of the robotic surgical system of FIG. 1, including a distal portion of a ureteroscope.

FIG. 4 depicts a schematic view of an example of an articulating elongate member that may be used with the robotic surgical system of FIG. 1.

FIG. 5 depicts a cross-sectional end view of the elongate member of FIG. 4, taken along line 5-5 of FIG. 4.

FIG. 6 depicts a cross-sectional end view of the elongate member of FIG. 4, taken along line 6-6 of FIG. 4.

FIG. 7 depicts a cross-sectional end view of the elongate member of FIG. 4, taken along line 7-7 of FIG. 4.

FIG. 8 depicts a cross-sectional end view of another example of an articulating elongate member that may be used with the robotic surgical system of FIG. 1.

FIG. 9 depicts an enlarged cross-sectional end view of the elongate member of FIG. 8, showing the region A from FIG. 8.

FIG. 10 depicts a partial view of another example of an articulating elongate member that may be used with the robotic surgical system of FIG. 1, with certain features shown in transparency to reveal internal components.

FIG. 11 depicts a cross-sectional end view of the elongate member of FIG. 4, taken along line 11-11 of FIG. 10.

FIG. 12 depicts a cross-sectional end view of the elongate member of FIG. 4, taken along line 12-12 of FIG. 10.

FIG. 13 depicts a perspective view of components of an apparatus that may be used to manufacture an elongate member having a braided structure.

FIG. 14 depicts a perspective view of components of another apparatus that may be used to manufacture an elongate member having a braided structure.

FIG. 15 depicts an enlarged perspective view of a distal portion of the apparatus of FIG. 14.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. It will be further appreciated that, for convenience and clarity, spatial terms such as “side,” “upwardly,” and “downwardly” also are used herein for reference to relative positions and directions. Such terms are used below with reference to views as illustrated for clarity and are not intended to limit the invention described herein.

Aspects of the present examples described herein may be integrated into a robotically-enabled medical system, including as a robotic surgical system, capable of performing a variety of medical procedures, including both minimally invasive, such as laparoscopy, and non-invasive, such as endoscopy, procedures. Among endoscopy procedures, the robotically-enabled medical system may be capable of performing bronchoscopy, ureteroscopy, gastroscopy, etc.

In addition to performing the breadth of procedures, the robotically-enabled medical system may provide additional benefits, such as enhanced imaging and guidance to assist the medical professional. Additionally, the robotically-enabled medical system may provide the medical professional with the ability to perform the procedure from an ergonomic position without the need for awkward arm motions and positions. Still further, the robotically-enabled medical system may provide the medical professional with the ability to perform the procedure with improved ease of use such that one or more of the instruments of the robotically-enabled medical system may be controlled by a single operator.

### I. EXAMPLE OF ROBOTICALLY-ENABLED MEDICAL SYSTEM

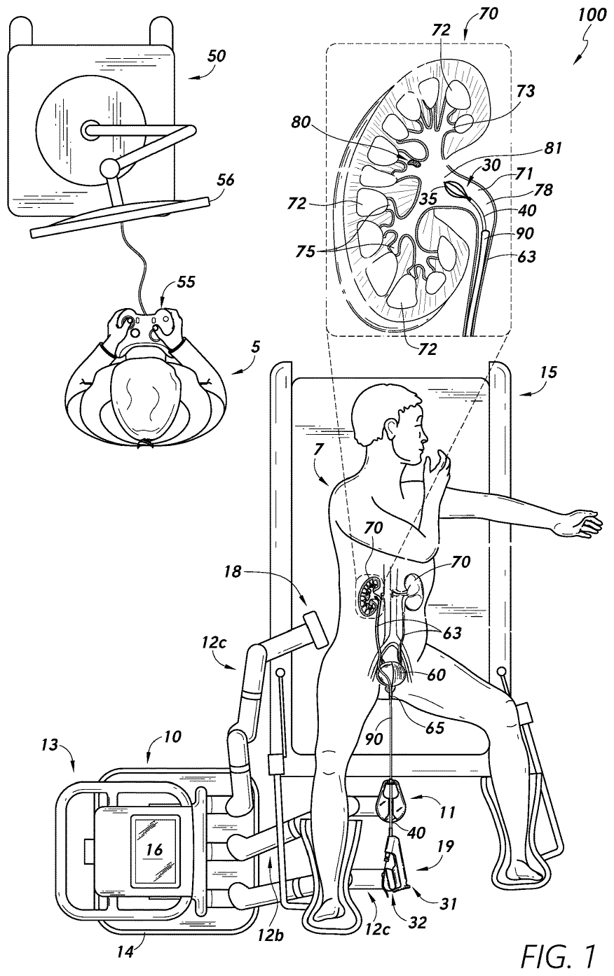

FIG. 1 shows an example medical system (100) for performing various medical procedures in accordance with aspects of the present disclosure. The medical system (100) may be used for, for example, endoscopic (e.g., ureteroscopic) procedures. Certain ureteroscopic procedures involve the treatment/removal of kidney stones. Although the system (100) of FIG. 1 is presented in the context of a ureteroscopic procedure, it should be understood that the principles disclosed herein may be implemented in any type of endoscopic (e.g., bronchial, gastrointestinal, etc.) and/or percutaneous procedure.

The medical system (100) of the present includes a robotic system (10) (e.g., mobile robotic cart) that is configured to engage with and/or control one or more medical instruments (e.g., ureteroscope (40), basketing system (30), etc.) via one or more robotic arms (12) to perform a direct-entry procedure on a patient (7). In some versions, the robotic system (10) and/or control system (50) is/are configured to receive images and/or image data from the scope (40) representing internal anatomy of the patient (7), namely the urinary system with respect to the particular depiction of FIG. 1, and/or display images based thereon.

It should be understood that the direct-entry instrument(s) operated through systems (10, 50) may include any type of medical instrument or combination of instruments, including an endoscope (such as a ureteroscope (40)), catheter (such as a steerable or non-steerable catheter), nephroscopes, laparoscope, basketing systems (30), and/or other type of medical instrument(s). The various scope-type instruments disclosed herein, such as the scope (40) of the system (100), may be configured to navigate within the human anatomy, such as within a natural orifice or lumen of the human anatomy. The terms “scope” and “endoscope” are used herein according to their broad and ordinary meanings; and may refer to any type of elongate medical instrument having image generating, viewing, and/or capturing functionality and configured to be introduced into any type of organ, cavity, lumen, chamber, or space of a body. A scope may include, for example, a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), colonoscope (e.g., for accessing the colon and/or rectum), borescope, and so on. Scopes/endoscopes, in some instances, may comprise a rigid or flexible tube, and may be dimensioned to be passed within an outer sheath, catheter, introducer, or other lumen-type device, or may be used without such devices.

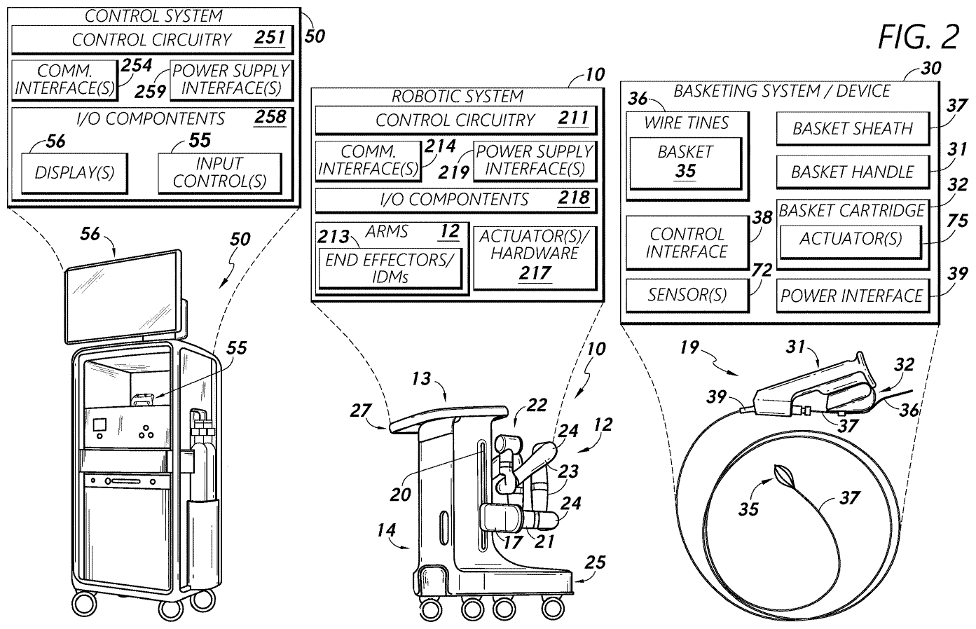

The medical system (100) of the present example further includes a control system (50), a table (15), and an electromagnetic (EM) field generator (18). Table (15) is configured to hold the patient (7). EM field generator (18) may be held by one or more of the robotic arms (12) of the robotic system (10) or may be a stand-alone device. As shown in FIGS. 1-2, control system (50) of the present example includes various input/output (I/O) components (258) configured to assist the physician (5) or others in performing a medical procedure. For example, the I/O components (258) may be configured to allow for user input to control/navigate the scope (40) and/or basketing system (30) within the patient (7). I/O components (258) of the present example include a controller (55) that is configured to receive user input from the operator; and a display (56) that is configured to present certain information to assist the operator. Controller (55) may take any suitable form, including but not limited to one or more buttons, keys, joysticks, handheld controllers (e.g., video-game-type controllers), computer mice, trackpads, trackballs, control pads, and/or sensors (e.g., motion sensors or cameras) that capture hand gestures and finger gestures, touchscreens, etc.

As also shown in FIG. 2, control system (50) of the present example includes a communication interface (254) that is operable to provide a communicative interface between control system (50) and robotic system (10), basketing system (30), scope (40), and/or other components. Communications via communication interface (254) may include data, commands, electrical power, and/or other forms of communication. Communication interface (254) may also be configured to provide communication via wire, wirelessly, and/or other modalities. Control system (50) also includes a power supply interface (259), which may receive power to drive control system (50) via wire, battery, and/or any other suitable kind of power source. A control circuitry (251) of control system (50) may provide signal processing and execute control algorithms to achieve the functionality of medical system (100) as described herein.

The control system (50) may also communicate with the robotic system (10) to receive position data therefrom relating to the position of the distal end of the scope (40), access sheath (90), or basketing device (30). Such positional data relating to the position of the scope (40), access sheath (90), or basketing device (30) may be derived using one or more electromagnetic sensors associated with the respective components. Moreover, in some versions, the control system (50) may communicate with the table (15) to position the table (15) in a particular orientation or otherwise control the table (15). The control system (50) may also communicate with the EM field generator (18) to control generation of an EM field in an area around the patient (7).

As noted above and as shown in FIGS. 1-2, robotic system (10) includes robotic arms (12) that are configured to engage with and/or control scope (40) and/or the basketing system (30) to perform one or more aspects of a procedure. It should be understood that robotic arms (12) may be coupled to different instruments than what is shown in FIG. 1; and in some scenarios, one or more of the robotic arms (12) may not be utilized or coupled to a medical instrument. Each robotic arm (12) includes multiple arm segments (23) coupled to joints (24), which may provide multiple degrees of movement/freedom. In the example of FIG. 1, the robotic system (10) is positioned proximate to the patient's legs and the robotic arms (12) are actuated to engage with and position the scope (40) for access into an access opening, such as the urethra (65) of the patient (7). When the robotic system (10) is properly positioned, the scope (40) may be inserted into the patient (7) robotically using the robotic arms (12), manually by the physician (5), or a combination thereof. A scope-driver instrument coupling (11) (e.g., instrument device manipulator (IDM)) may be attached to the distal portion of one of the arms (12b) to facilitate robotic control/advancement of the scope (40). Another (12c) of the arms may include an instrument coupling/manipulator (19) that is configured to facilitate advancement and operation of the basketing device (30). The scope (40) may include one or more working channels through which additional tools, such as lithotripters, basketing devices, forceps, etc., may be introduced into the treatment site.

The robotic system (10) may be coupled to any component of the medical system (100), such as the control system (50), the table (15), the EM field generator (18), the scope (40), the basketing system (30), and/or any type of percutaneous-access instrument (e.g., needle, catheter, nephroscope, etc.). As noted above, robotic system (10) may be communicatively coupled with control system (50) via communication interfaces (214, 254). Robotic system (10) also includes a power supply interface (219), which may receive power to drive robotic system (10) via wire, battery, and/or any other suitable kind of power source. In addition, robotic system (10) of the present example includes various input/output (I/O) components (218) configured to assist the physician (5) or others in performing a medical procedure. Such I/O components (218) may include any of the various kinds of I/O components (258) described herein in the context of control system (50). In addition, or in the alternative, I/O components (218) of robotic system (10) may take any suitable form (or may be omitted altogether).

Robotic system (10) of the present example generally includes a column (14), a base (25), and a console (13) at the top of the column (14). The column (14) may include one or more arm supports (17) (also referred to as a “carriage”) for supporting the deployment of the one or more robotic arms (12) (three shown in FIG. 2). The arm support (17) may include individually-configurable arm mounts that rotate along a perpendicular axis to adjust the base of the robotic arms (12) for desired positioning relative to the patient. In some versions, the arm support (17) may be connected to the column (14) through slots (20) that are positioned on opposite sides of the column (14) to guide vertical translation of the arm support (17) along column (14). The robotic arms (12) of the present example generally comprise robotic arm bases (21) and end effectors (22), separated by a series of linking arm segments (23) that are connected by a series of joints (24), each joint comprising one or more independent actuators (217). Each actuator (217) may comprise an independently-controllable motor. I/O components (218) may be positioned at the upper end of column (14). Console (13) also includes a handle (27) to assist with maneuvering and stabilizing robotic system (10).

The end effector (213) of each of the robotic arms (12) may include an instrument device manipulator (IDM), which may be attached using a mechanism changer interface (MCI). In some versions, the IDM (213) may be removed and replaced with a different type of IDM (213), for example, a first type (11) of IDM (213) may manipulate a scope (40), while a second type (19) of IDM (213) may manipulate a basketing system (30). Another type of IDM (213) may be configured to hold an electromagnetic field generator (18). An MCI may provide power and control interfaces (e.g., connectors to transfer pneumatic pressure, electrical power, electrical signals, and/or optical signals from the robotic arm (12) to the IDM (213). The IDMs (213) may be configured to manipulate medical instruments (e.g., surgical tools/instruments), such as the scope (40), using techniques including, for example, direct drives, harmonic drives, geared drives, belts and pulleys, magnetic drives, and the like.

The system (100) may include certain control circuitry configured to perform certain of the functionality described herein, including the control circuitry (211) of the robotic system (10) and the control circuitry (251) of the control system (50). That is, the control circuitry of the system (100) may be part of the robotic system (10), the control system (50), or some combination thereof. The term “control circuitry” is used herein according to its broad and ordinary meaning, and may refer to any collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further include one or more circuit substrates (e.g., printed circuit boards), conductive traces and vias, and/or mounting pads, connectors, and/or components. Control circuitry referenced herein may further comprise one or more storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in versions in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The control circuitry (211, 251) may comprise computer-readable media storing, and/or configured to store, hard-coded and/or operational instructions corresponding to at least some of the steps and/or functions illustrated in one or more of the present figures and/or described herein. Such computer-readable media may be included in an article of manufacture in some instances. The control circuitry (211, 251) may be entirely locally maintained/disposed or may be remotely located at least in part (e.g., communicatively coupled indirectly via a local area network and/or a wide area network).

In some versions, for example, the physician (5) may provide input to the control system (50) and/or robotic system (10); and in response to such input, control signals may be sent to the robotic system (10) to manipulate the scope (40) and/or catheter basketing system (30). The control system (50) may include one or more display devices (56) to provide various information regarding a procedure. For example, the display(s) (56) may provide information regarding the scope (40) and/or basketing system (30). The control system (50) may receive real-time images that are captured by the scope (40) and display the real-time images via the display(s) (56).

As shown in FIG. 2, the basketing device (30) of the present example includes a basket (35) formed of one or more wire tines (36) disposed within a basketing sheath (37) over a length thereof, where the tines project from a distal end of the sheath (37) to form the basket (35). The tines (36) further extend from the proximal end of the sheath (37) and are slidable within the basketing sheath (37). The tines (36) and the sheath (37) may be coupled to respective actuators (75) of a basket cartridge component (32). The basket cartridge (32) may be physically and/or communicatively coupled to a handle portion/component (31) of the basketing system (30). The handle component (31) may be configured to be used to assist in basketing control either manually or through robotic control. The basketing system (30) may be powered through a power interface (39) and/or controlled through a control interface (38), each or both of which may interface with a robotic arm/component of the robotic system (10). The basketing system (30) may further comprise one or more sensors (72), such as pressure and/or other force-reading sensors, which may be configured to generate signals indicating forces experienced at/by one or more of the actuators (75) and/or other couplings of the basketing system (30).

In an example of a use case, if the patient (7) has a kidney stone (80) located in the kidney (70), the physician may execute a procedure to remove the stone (80) through the urinary tract (65, 60, 63). In particular, and as shown in FIG. 1, the physician may operate medical system (100) to achieve direct entry of the scope (40) into the urinary tract (65, 60, 63) of the patient (7) via the urethra (65). The physician (5) may interact with the control system (50) and/or the robotic system (10) to cause/control the robotic system (10) to advance and navigate the scope (40) from the urethra (65), through the bladder (60), up the ureter (63), and into the renal pelvis (71) and/or calyx network of the kidney (70) where the stone (80) is located. The physician (5) may further interact with the control system (50) and/or the robotic system (10) to cause/control the advancement of a basketing device (30) through a working channel of the scope (40), where the basketing device (30) is configured to facilitate capture and removal of a kidney stone. The control system (50) may provide information via the display(s) (56) that is associated with the medical instrument (40), such as real-time endoscopic images captured therewith, and/or other instruments of the system (100), to assist the physician (5) in navigating/controlling such instrumentation.

In the present example, a ureteral access sheath (90) is disposed within the urinary tract (65, 60, 63) to an area near the kidney (70). The scope (40) may be passed through the ureteral access sheath (90) to gain access to the internal anatomy of the kidney (70), as shown. Once at the site of the kidney stone (80) (e.g., within a target calyx (73) of the kidney (70) through which the stone (80) is accessible), the scope (40) may be used to channel/direct the basketing device (30) to the target location. Once the stone (80) has been captured in the distal basket portion (35) of the basketing device (30), the utilized ureteral access path may be used to extract the kidney stone (80) from the patient (7).

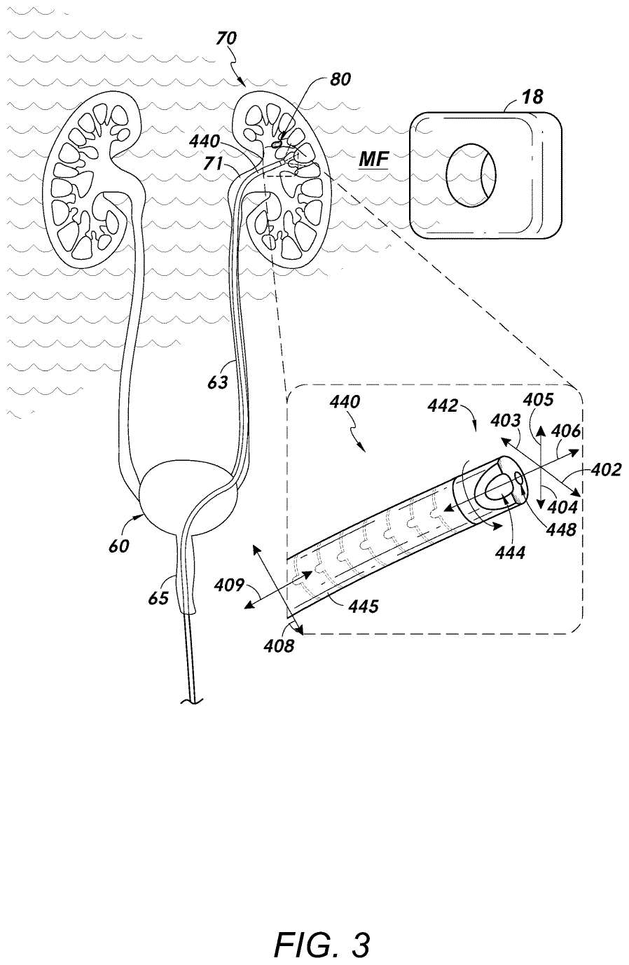

FIG. 3 shows an example of a scope (440) that may be used as scope (40) described above. Scope (440) of this example includes a working channel (444) for deploying medical instruments (e.g., lithotripters, basketing system (30), forceps, etc.), irrigation, and/or aspiration to an operative region at a distal end of the scope (440). The scope (440) may be articulated, such as with respect to at least a distal portion of the scope (440), so that the scope (440) can be steered within the human anatomy. In some versions, the scope (440) is configured to be articulated with, for example, five degrees of freedom, including XYZ coordinate movement, as well as pitch and yaw. In some versions, the scope (440) provides six degrees of freedom, including X, Y, and Z ordinate positions, as well as pitch, roll, and yaw. Position sensor(s) of the scope (440) may likewise have similar degrees of freedom with respect to the position information they produce/provide. As shown in FIG. 3, the tip (442) of the scope (440) may be oriented with zero deflection relative to a longitudinal axis (406) thereof (also referred to as a “roll axis”).

In the present example, the scope (440) can accommodate wires and/or optical fibers to transfer signals to/from an optical assembly and a distal end (442) of the scope (440), which can include an imaging device (448), such as an optical camera. The imaging device (448) may be used to capture images of an internal anatomical space, such as a target calyx/papilla of the kidney (70). The scope (440) may further be configured to accommodate optical fibers to carry light from proximately-located light sources, such as light-emitting diodes, to the distal end (442) of the scope (440). The distal end (442) of the scope (440) may include ports for light sources to illuminate an anatomical space when using the imaging device (448). The imaging device (448) may comprise an optical fiber, fiber array, and/or lens; or a light-emitting diode at distal end (442). The optical components of imaging device (448) move along with the distal end (442) of the scope (440), such that movement of the distal end (442) of the scope (440) results in changes to the images captured by the imaging device(s) 448.

To capture images at different orientations of the tip (442), robotic system (10) may be configured to deflect the tip (442) on a positive yaw axis (402), negative yaw axis (403), positive pitch axis (404), negative pitch axis (405), or roll axis (406). The tip (442) or body (445) of the scope (442) may be elongated or translated in the longitudinal axis (406), x-axis (408), or y-axis (409). The scope (440) may include a reference structure (not shown) to calibrate the position of the scope (440). For example, robotic system (10) and/or control system (50) may measure deflection of the scope (440) relative to the reference structure. The reference structure may be located, for example, on a proximal end of the endoscope (440) and may include a key, slot, or flange.

A robotic arm (12) of robotic system (10) may be configured/configurable to manipulate the scope (440) as described above. Such manipulation may be performed by actuating one or more elongate members such as one or more pull wires (e.g., pull or push wires), cables, fibers, and/or flexible shafts. For example, robotic arms (12) may be configured to actuate multiple pull wires (not shown) coupled to the scope (440) to deflect the tip (442) of the scope (440). Pull wires may include any suitable or desirable materials, such as metallic and non-metallic materials such as stainless steel, aramid fiber, tungsten, carbon fiber, and the like. In some versions, the scope (440) is configured to exhibit nonlinear behavior in response to forces applied by the elongate movement members. The nonlinear behavior may be based on stiffness and compressibility of the scope (440), as well as variability in slack or stiffness between different elongate movement members.

In some versions, the scope (440) includes at least one sensor that is configured to generate and/or send sensor position data to another device. The sensor position data can indicate a position and/or orientation of the scope (440) (e.g., the distal end (442) thereof) and/or may be used to determine/infer a position/orientation of the scope (440). For example, a sensor (sometimes referred to as a “position sensor”) may include an electromagnetic (EM) sensor with a coil of conductive material or other form of an antenna. In some versions, the position sensor is positioned on the distal end (442) of scope (440), while in other embodiments the sensor is positioned at another location on scope (440).

As shown in FIG. 3, EM field generator (18) is configured to broadcast an alternating EM field 90 that is detected by the EM position sensor of scope (440). The alternating magnetic field (MF) induces small currents in coils of the EM position sensor, which may be analyzed to determine a distance and/or angle/orientation between the EM position sensor and the EM field generator (18). It should be understood that scope (440) may include other types of sensors, such as a shape sensing fiber, accelerometer(s), gyroscope(s), satellite-based positioning sensor(s) (e.g., global positioning system (GPS) sensors), radio-frequency transceiver(s), and so on. In the present example, the EM position sensor of scope (440) provides sensor data to control system (50), which is then used to determine a position and/or an orientation of scope (440).

In some variations, any of the features and aspects described above may be constructed and operable in accordance with at least some of the teachings of U.S. Pat. No. 11,737,663, entitled “Target Anatomical Feature Localization,” issued Aug. 29, 2023, the disclosure of which is incorporated by reference herein, in its entirety; U.S. Pub. No. 2021/0369384, entitled “Stuck Instrument Management,” published Dec. 2, 2021, the disclosure of which is incorporated by reference herein, in its entirety; U.S. Pub. No. 2021/0401527, entitled “Robotic Medical Systems Including User Interfaces with Graphical Representations of User Input Devices,” published Dec. 30, 2021, the disclosure of which is incorporated by reference herein, in its entirety; and/or U.S. Pub. No. 2022/0096183, entitled “Haptic Feedback for Aligning Robotic Arms,” published Mar. 31, 2022, the disclosure of which is incorporated by reference herein, in its entirety.

### II. EXAMPLES OF ALTERNATIVE ARTICULATION ASSEMBLIES FOR ELONGATE MEMBER

As noted above, robotic system (10) may include one or more articulating elongate instruments, such as scope (40), access sheath (90), scope (440), catheters, and/or other kinds of elongate instruments. Some elongate instruments may include articulating sections that are formed of a laser-cut tubular structure, where the laser-cut pattern is configured to promote bending of the tubular structure. Such laser-cut tubular structures may provide undesirable expense to a manufacturing process. Some alternative configurations may include the use of a braided structure in an articulating section of an elongate instrument. However, some such braided articulating section structures may tend to undergo a greater degree of longitudinal compression during articulation. Another design consideration for articulating elongate members is force isolation. In the absence of sufficient force isolation, where an operator only wishes to articulate a distal portion of the elongate member, a proximal portion of the elongate member may buckle or have some other undesired effect. A Bowden tube arrangement may be used to effectively isolate the proximal portion of the elongate member from articulation related forces, thereby avoiding undesired buckling or other undesired effects. However, such a Bowden tube arrangement may tend to add undesired cost and/or necessitate an undesired increase in wall thickness (i.e., reducing cross-sectional efficiency) of the elongate member.

In view of the foregoing, it may therefore be desirable to provide a structure for articulating section of an elongate instrument that resists compression like a laser-cut tubular structure, while providing reduced manufacturing costs like a braided structure, and while also providing force isolation without the potential drawbacks of Bowden tubes. The following describes several examples of elongate members with articulating sections constructed to provide lateral flexibility and reduced manufacturing costs while also avoiding longitudinal compression during articulation and providing force isolation to prevent inadvertent buckling or other undesired effects from articulation.

A. Example of Elongate Member with Pair of Helically Oriented Stiffening Wires

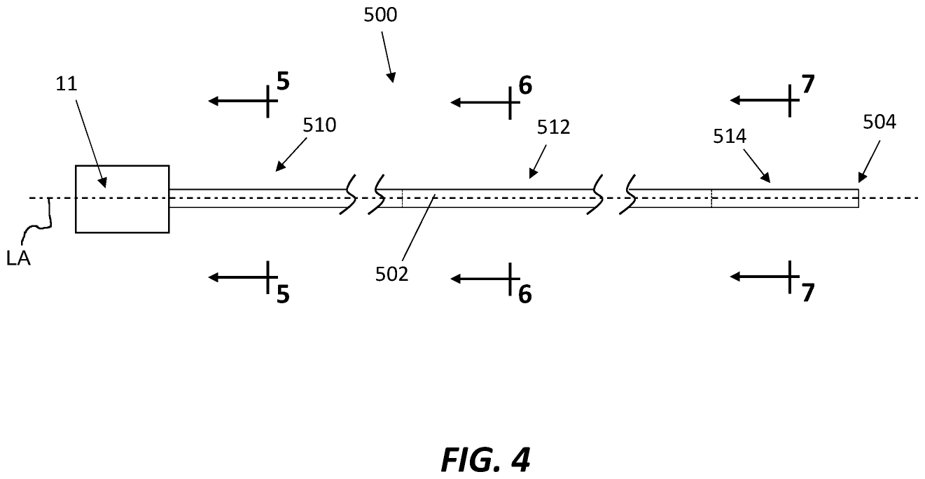

FIGS. 4-7 show an example of an articulating elongate member (500) that may be used with robotic surgical system (10). By way of example only, elongate member (500) may represent a variation of scope (40), access sheath (90), or scope (440). Alternatively, elongate member (500) may take the form of a catheter and/or any other suitable kind of elongate instrument. Elongate member (500) of the present example includes a body (502) having a proximal portion (510), a medial portion (512), and a distal portion (514). In the present example, at least distal portion (514) is operable to articulate, such that distal end (504) of elongate member (500) may be deflected laterally away from and toward a central longitudinal axis (LA) (e.g., defined by proximal portion (510)). In some versions, elongate member (500) is operable to articulate at one or more different regions along the length of elongate member (500). For instance, distal portion (514) may include one or more articulation sections, medial portion (512) may include one or more articulation sections, and/or proximal portion (510) may include one or more articulation sections.

Proximal portion (510) is coupled with instrument coupling (11) of robotic surgical system (10), such that robotic surgical system (10) is operable to drive elongate member (500) via instrument coupling (11). By way of example only, robotic surgical system (10) may be operable to drive translation along the central longitudinal axis (LA), rotation (e.g., spinning about the central longitudinal axis (LA)), articulation, and/or other forms of movement of/by elongate member (500).

Distal end (504) of the present example may include one or more openings through which one or more additional instruments may exit into a surgical space or other anatomical region within a patient. Distal end (504) may also include one or more imaging devices, such as an imaging device (448), that may take the form of one or more cameras, one or more optical fibers with corresponding lenses, etc. Distal end (504) may also include one or more illuminating elements, such as one or more integral light-emitting diodes, one or more lenses optically coupled with corresponding optical fibers, etc. In some versions, distal end (504) includes an end effector that is operable to perform one or more operations on tissue, such as grasping, cutting, suturing, sealing (e.g., via RF energy or ultrasonic energy), stapling, etc.

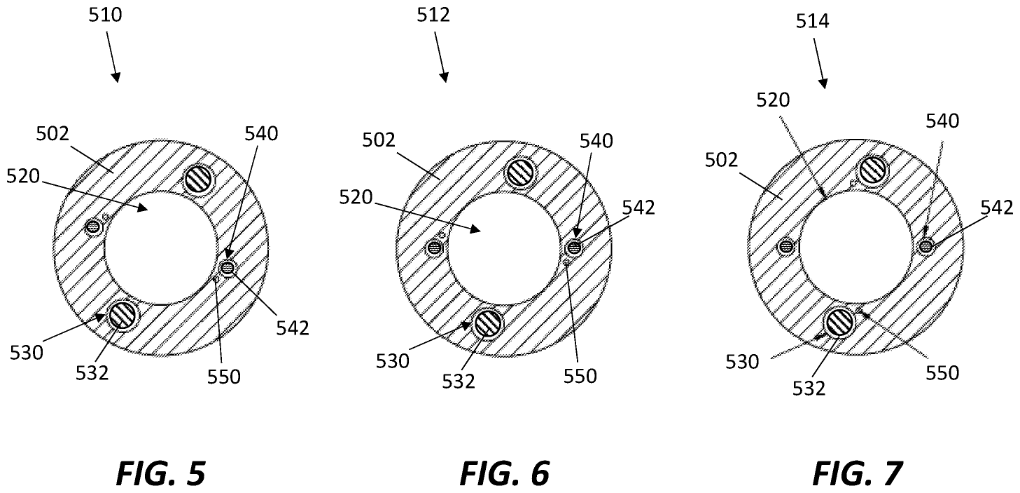

As shown in FIG. 5-7, body (502) of the present example defines a working channel (520), a pair of electrical wire lumens (530), and a pair of tendon lumens (540). Body (502) may comprise polyether block amide (PEBA) and/or any other suitable kind(s) of material(s). Working channel (520) is configured to slidably receive another instrument. By way of example only, basket (35) and basketing sheath (37) of basketing device (30) may be advanced distally via working channel (520). Working channel (520) may extend all the way to distal end (504), where working channel (520) may terminate in a distal opening allowing an instrument that is disposed in working channel (520) to exit distally from elongate member (500). In some versions, working channel (520) includes a liner that is configured to reduce friction and thereby promote slidability through working channel (520). By way of example only, such a liner may include polytetrafluoroethylene (PTFE), polyimide, and/or any other suitable kind(s) of material(s).

In the present example, electrical wire lumens (530) are positioned approximately 180 degrees apart from each other about the central longitudinal axis (LA). Each electrical wire lumen (530) contains at least one corresponding electrical wire (532) in the present example. Such electrical wires (532) may be configured to provide electrical communication to/from one or more components at distal end (504), including but not limited to one or more cameras, one or more light sources, one or more position sensors, one or more electrodes, etc. Such electrical wires (532) may also be coupled with one or more corresponding components of robotic system (10), control system (50) and/or other devices. In some versions, each electrical wire lumen (530) includes a liner such as polytetrafluoroethylene (PTFE), polyimide, and/or any other suitable kind(s) of material(s). While two electrical wire lumens (530) are shown in the present example, other variations of elongate member (500) may include just one wire lumen (530) or more than two electrical wire lumens (530).

Tendon lumens (540) are also positioned approximately 180 degrees apart from each other about the central longitudinal axis (LA) in the present example. Each tendon lumen (540) contains a corresponding tendon (542) in the present example. Each tendon (542) has a distal end that is fixedly secured to a corresponding portion of elongate member (500). In some versions, one tendon (542) has a distal end that is fixedly secured at or near distal end (504) of elongate member (500) to provide articulation of distal portion (514); while another tendon (542) has a distal end that is fixedly secured at or near a transition from medial portion (512) to distal portion (514), to provide articulation of medial portion (512). In some other versions, each tendon (542) has a distal end that is fixedly secured at or near distal end (504) of elongate member (500) to provide bidirectional articulation of distal portion (514).

By way of example only, each tendon (542) may comprise a pull wire, a drive band, a single-strand cable, a multi-strand cable, one or more metals, one or more fibers, and/or any other suitable component that is operable to communicate a pulling force along the length of elongate member (500), to thereby provide articulation of elongate member (500), without substantially stretching. Such tendons (542) may also be coupled with instrument coupling (11) of robotic surgical system (10), such that robotic surgical system (10) is operable to drive tendons (542) via instrument coupling (11). In some versions, each tendon lumen (540) includes a liner such as polytetrafluoroethylene (PTFE), polyimide, and/or any other suitable kind(s) of material(s). While two tendon lumens (540) are shown in the present example, other variations of elongate member (500) may include just one tendon lumen (540) or more than two tendon lumens (540).

Elongate member (500) of this example further includes a pair of stiffening wires (550) that extend along the length of body (502). Stiffening wires (550) may also be referred to as neutral axis wires. Stiffening wires (550) are positioned approximately 180 degrees apart from each other about the central longitudinal axis (LA). The distal end of each stiffening wire (550) is fixed at distal end (504) of body (502); while the proximal end of each stiffening wire (550) is also fixed relative to body (502) such that stiffening wires (550) are not configured to translate longitudinally. Each stiffening wire (550) is formed of a non-compressible material having a high tensile strength. By way of example only, each stiffening wire (550) may comprise stainless steel, tungsten, hyten, aramid fiber, and/or any other suitable material(s). Stiffening wires (550) may be in the form of a monofilament, a braided structure, a bundle of filaments, or any other suitable form.

As shown in FIGS. 5-6, stiffening wires (550) are positioned in close angular proximity to corresponding tendons (542), such that stiffening wires (550) effectively run alongside corresponding tendons (542), along proximal portion (510) and medial portion (512) of elongate member (500). As shown in FIG. 7, stiffening wires (550) are positioned in close angular proximity to corresponding electrical wires (532), such that stiffening wires (550) effectively run alongside corresponding electrical wires (532), along distal portion (514) of elongate member (500). Stiffening wires (550) are angularly offset from tendons (542) by approximately 90 degrees about the central longitudinal axis (LA) in distal portion (514).

With stiffening wires (550) effectively running alongside tendons (542) along proximal portion (510) and medial portion (512) of elongate member (500), and with stiffening wires (550) being angularly offset from tendons (542) by approximately 90 degrees about the central longitudinal axis (LA) in distal portion (514) of elongate member (500), this varying spatial relationship between stiffening wires (550) and tendons (542) may provide articulation performance that varies along the length of elongate member (550). In particular, since stiffening wires (550) effectively run alongside tendons (542) along proximal portion (510) and medial portion (512) of elongate member (500), neither proximal portion (510) and medial portion (512) of elongate member (500) will articulate in response to tension being applied to either tendon (542). In other words, stiffening wires (550) will effectively prevent tension in tendons (542) from resulting in lateral deflection of proximal portion (510) and medial portion (512) of elongate member (500) in regions of body (502) where since stiffening wires (550) effectively run alongside tendons (542). Stiffening wires (550) thus effectively define the neutral axis or neutral plane, providing a bending moment of essentially zero. Stiffening wires (550) may also prevent tension that is applied to tendons (542) from resulting in compression of body (502) along proximal portion (510) and medial portion (512).

Since stiffening wires (550) are angularly offset from tendons (542) by approximately 90 degrees about the central longitudinal axis (LA) in distal portion (514) of elongate member (500), stiffening wires (550) do not impede lateral deflection of distal portion (514) of elongate member (500) when tension is applied to either tendon (542). Stiffening wires (550) thus provide force isolation relative to tendons (542), based on the longitudinal portion (510, 512, 514) of elongate member (500).

It should also be understood that, since stiffening wires (550) effectively run alongside electrical wires (532) along distal portion (514), this spatial relationship between stiffening wires (550) and electrical wires (532) may effectively prevent cyclic elongation and compression of electrical wires (532) that might otherwise occur in the absence of stiffening wires (550) during articulation of distal portion (514). In other words, stiffening wires (550) may improve the durability of electrical wires (532). It should also be understood that since stiffening wires (550) are positioned along the neutral plane (relative to the articulation plane) along distal portion (514); and that electrical wires (532) are substantially near the same neutral plane, such that this positioning may also tend to minimize mechanical wear on electrical wires (532) during articulation of distal portion (514).

Also in the present example, lumens (530, 540) and stiffening wires (550) extend along respective helical paths about the central longitudinal axis (LA) along proximal portion (510) and medial portion (512). By way of example only, such helical paths may have a pitch ranging from approximately 0.125 winds per inch to approximately 0.750 winds per inch; or more particularly may be approximately 0.2 winds per inch. Alternatively, any other suitable pitch may be provided. In some cases, having tendon lumens (540) and tendons (542) extend along helical paths along proximal portion (510) and medial portion (512) may prevent tendons (742) from causing inadvertent articulation in distal portion (514) when proximal portion (510) or medial portion (512) is incidentally bent during use of elongate member (500). For instance, if tendons (542) were to follow paths that are parallel with the central longitudinal axis (LA) along proximal portion (510) and medial portion (512), and if elongate member (500) were to traverse a tortuous path in the patient anatomy such that proximal portion (510) or medial portion (512) were bent, such bending of proximal portion (510) or medial portion (512) were bent may tend to impart a pulling force on one of tendons (542), which may in turn cause undesired articulation at distal portion (514). The helical orientation of stiffening wires (550) extend along proximal portion (510) and medial portion (512) may also increase the column strength and tensile strength of elongate member (500) along proximal portion (510) and medial portion (512); while still allowing proximal portion (510) and medial portion (512) to substantially bend laterally to conform to tortuous anatomical structures in the patient.

At a transition from medial portion (512) to distal portion (514), lumens (530, 540) and stiffening wires (550) extend along respective paths that are parallel to the central longitudinal axis (LA); and continue along these parallel paths through distal portion (514). Electrical wire lumens (530) maintain 180 degree angular spacing from each other along all three portions (510, 512, 514) of elongate member. Tendon lumens (540) also maintain 180 degree angular spacing from each other along all three portions (510, 512, 514) of elongate member. Stiffening wires (550) also maintain 180 degree angular spacing from each other along all three portions (510, 512, 514) of elongate member. However, at the transition from medial portion (512) to distal portion (514), stiffening wires (550) each traverse a 90 degree angular path about the central longitudinal axis (LA), to transition from effectively running alongside tendon lumens (540) to effectively running alongside electrical wire lumens (530).

In some other versions, lumens (530, 540) and stiffening wires (550) do not extend along respective helical paths about the central longitudinal axis (LA) along proximal portion (510) and medial portion (512). In such versions, lumens (530, 540) and stiffening wires (550) may extend along respective paths that are parallel to the central longitudinal axis (LA) along the proximal portion (510), medial portion (512), and distal portion (514). Even in these versions, at the transition from medial portion (512) to distal portion (514), stiffening wires (550) may still traverse a 90 degree angular path about the central longitudinal axis (LA), to transition from effectively running alongside tendon lumens (540) to effectively running alongside electrical wire lumens (530).

In the example provided above, elongate member (500) is operable to articulate bi-directionally along a single articulation plane. Some variations may provide articulation along two or more planes. In some such variations, elongate member (500) may be configured to effectively articulate along two or more planes without necessarily including additional tendon lumens (540), tendons (542), and corresponding stiffening wires (550). For instance, a variation of elongate member (500) may be configured to effectively articulate along two or more planes by simply allowing elongate member (500) to roll about the central longitudinal axis (LA), to thereby re-orient the plane of articulation about the central longitudinal axis (LA). Such re-oriented planes of articulation may effectively serve as multiple different planes of articulation. In some such variations, an additional actuator (not shown) is provided to drive rotary motion of elongate member (500) about the central longitudinal axis (LA).

In some other variations, to provide multi-plate articulation, additional tendon lumens (540), tendons (542), and corresponding stiffening wires (550) may be provided. Such additional lumens (540), tendons (542), and corresponding stiffening wires (550) may be angularly offset from the lumens (540), tendons (542), and corresponding stiffening wires (550) described above by 90 degrees about the central longitudinal axis (LA). The addition of such lumens (540), tendons (542), and corresponding stiffening wires (550) may further warrant repositioning of electrical wire lumens (530) and electrical wires (532). An example of a variation of elongate member (500) with additional tendons and stiffening wires is described in greater detail below with reference to elongate member (700).

B. Example of Elongate Member with Pair of Helically Oriented Stiffening wire and Coaxial Braided Structures

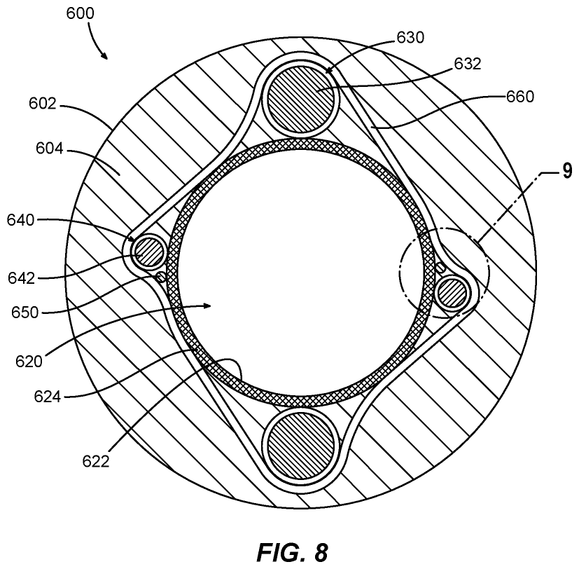

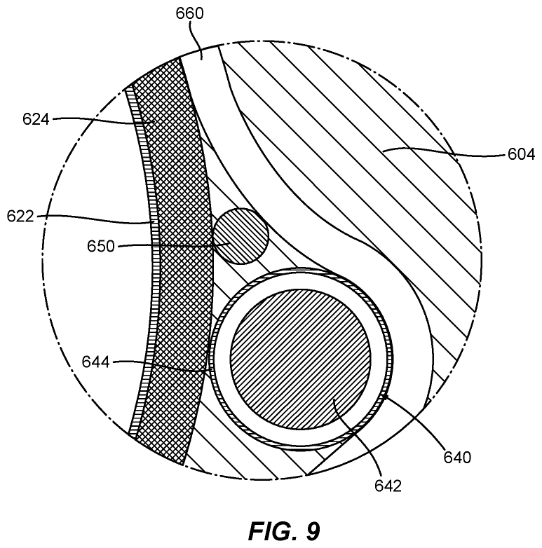

In some scenarios, it may be beneficial to provide additional structural integrity within an elongate member such as elongate member (500) described above. Depending on the material(s) used to form body (502), such additional structural integrity may be desirable to prevent tendons (542), stiffening wires (550), and/or other features of elongate member (500) from damaging body (502) (e.g., “cheese wire”-type of cutting into body (502)) during use of elongate member (500). It may further be desirable to include one or more features that provide such structural integrity without substantially compromising the flexibility of elongate member (500), without substantially increasing the outer diameter of elongate member (500), and without substantially reducing the inner diameter of working channel (520). To these ends, FIGS. 8-9 show an example of an elongate member (600) representing a variation of elongate member (500). Elongate member (600) may be constructed and operable like elongate member (500) except as otherwise described below.

Elongate member (600) of the present example includes a body (602) that defines a working channel (620), a pair of electrical wire lumens (630), and a pair of tendon lumens (640). Body (602) may comprise polyether block amide (PEBA) and/or any other suitable kind(s) of material(s). Working channel (620) is configured to slidably receive another instrument. By way of example only, basket (35) and basketing sheath (37) of basketing device (30) may be advanced distally via working channel (620). Like working channel (520) described above, working channel (620) may extend all the way to the distal end (not shown) of elongate member (600), where working channel (620) may terminate in a distal opening allowing an instrument that is disposed in working channel (620) to exit distally from elongate member (600). In the present example, working channel (620) includes a liner (622) that is configured to reduce friction and thereby promote slidability through working channel (620). By way of example only, liner (622) may include polytetrafluoroethylene (PTFE), polyimide, and/or any other suitable kind(s) of material(s).

In the present example, electrical wire lumens (630) are positioned approximately 180 degrees apart from each other about the central longitudinal axis (LA). Each electrical wire lumen (630) contains at least one corresponding electrical wire (632) in the present example. Such electrical wires (632) may be configured to provide electrical communication to/from one or more components at the distal end of elongate member (600), including but not limited to one or more cameras, one or more light sources, one or more position sensors, one or more electrodes, etc. Such electrical wires (632) may also be coupled with one or more corresponding components of robotic system (10), control system (50) and/or other devices. In some versions, each electrical wire lumen (630) includes a liner such as polytetrafluoroethylene (PTFE), polyimide, and/or any other suitable kind(s) of material(s). While two electrical wire lumens (630) are shown in the present example, other variations of elongate member (600) may include just one wire lumen (630) or more than two electrical wire lumens (630).

Tendon lumens (640) are also positioned approximately 180 degrees apart from each other about the central longitudinal axis (LA) in the present example. Each tendon lumen (640) contains a corresponding tendon (642) in the present example. Each tendon (642) has a distal end that is fixedly secured to a corresponding portion of elongate member (600). In some versions, one tendon (642) has a distal end that is fixedly secured at or near the distal end of elongate member (600) to provide articulation of a distal portion of elongate member (600); while another tendon (642) has a distal end that is fixedly secured at or near a transition from a medial portion of elongate member (600) to the distal portion of elongate member (600), to provide articulation of the medial portion. In some other versions, each tendon (642) has a distal end that is fixedly secured at or near the distal end of elongate member (600) to provide bidirectional articulation of the distal portion.

By way of example only, each tendon (642) may comprise a pull wire, a drive band, a single-strand cable, a multi-strand cable, one or more metals, one or more fibers, and/or any other suitable component that is operable to communicate a pulling force along the length of elongate member (600), to thereby provide articulation of elongate member (600), without substantially stretching. Such tendons (642) may also be coupled with instrument coupling (11) of robotic surgical system (10), such that robotic surgical system (10) is operable to drive tendons (642) via instrument coupling (11). In the present example, each tendon lumen (640) also includes a liner (644), which may comprise polytetrafluoroethylene (PTFE), polyimide, and/or any other suitable kind(s) of material(s). While two tendon lumens (640) are shown in the present example, other variations of elongate member (600) may include just one tendon lumen (640) or more than two tendon lumens (640).

Elongate member (600) of this example further includes a pair of stiffening wires (650) that extend along the length of body (602). Stiffening wires (650) are positioned approximately 180 degrees apart from each other about the central longitudinal axis (LA). The distal end of each stiffening wire (650) is fixed at the distal end of body (602); while the proximal end of each stiffening wire (650) is also fixed relative to body (602) such that stiffening wires (650) are not configured to translate longitudinally. Each stiffening wire (650) is formed of a non-compressible material having a high tensile strength. By way of example only, each stiffening wire (650) may comprise stainless steel, tungsten, hyten, aramid fiber, and/or any other suitable material(s). Stiffening wires (650) may be in the form of a monofilament, a braided structure, a bundle of filaments, or any other suitable form.

As shown in FIGS. 7-8, stiffening wires (650) are positioned in close angular proximity to corresponding tendons (642), such that stiffening wires (650) effectively run alongside corresponding tendons (642), along the proximal portion and medial portion of elongate member (600). While not shown, stiffening wires (650) may be positioned in close angular proximity to corresponding electrical wires (632), such that stiffening wires (650) effectively run alongside corresponding electrical wires (632), along the distal portion of elongate member (600). Stiffening wires (650) may be angularly offset from tendons (642) by approximately 90 degrees about the central longitudinal axis (LA) in the distal portion of elongate member (600). The relative positioning of stiffening wires (650), tendons (642), and electrical wires (632) in elongate member (600) may thus be substantially similar to the relative positioning of stiffening wires (550), tendons (542), and electrical wires (532) in elongate member (500). The functionality of stiffening wires (650) in elongate member (600) may also be substantially similar to the functionality of stiffening wires (550) in elongate member (500) as described above. Similarly, lumens (630, 640) and stiffening wires (650) may extend along respective helical paths about the central longitudinal axis (LA) along a proximal region of elongate member (600); and then along respective paths that are parallel with the central longitudinal axis (LA) along a distal region of elongate member (600).

Unlike elongate member (500), elongate member (600) of the present example further includes an inner braid (624) and an outer braid (660). Braids (600, 624) are positioned coaxially with body (602) in this example. Braids (600, 624) may comprise any suitable material and/or combination of materials, including but not limited to stainless steel wire, stainless steel cable, tungsten wire, tungsten cable, nitinol, liquid crystal polymer (LCP), aramid fiber, etc. Inner braid (624) is radially positioned outside of liner (622) of working channel (620). In some versions, inner braid (624) effectively defines working channel (620), with liner (622) being applied directly to the inner diameter of inner braid (624). Electrical wire lumens (630), tendon lumens (640), and stiffening wires (650) are radially positioned outside of inner braid (624). In scenarios where tendons (642) and/or stiffening wires (650) would otherwise tend to exert inwardly directed forces on body (502) (e.g., during bending of elongate member (600), particularly during driven articulation), inner braid (624) may effectively absorb such forces and thereby shield body (502) from any damage that might otherwise be caused to body (602) by tendons (642) and/or stiffening wires (650).

Outer braid (660) is radially positioned outside of electrical wire lumens (630), tendon lumens (640), stiffening wires (650). While inner braid (624) has a substantially circular cross-sectional profile as shown in FIG. 8, outer braid (660) does not have a substantially circular cross-sectional profile in this example. This is due to the process of manufacturing elongate member (600), where outer braid (660) is formed by wrapping the strands or wires of outer braid (660) about the exterior of inner braid (624), electrical wire lumens (630), tendon lumens (640), and stiffening wires (650). An example of such a process, and devices that may be used to perform such a process, are described in greater detail below.

In some versions, a proximal region of inner braid (624) has a braid density ranging from approximately 20 picks per inch (PPI) to approximately 150 PPI; ranging from approximately 50 PPI to approximately 200 PPI; ranging from approximately 20 PPI to approximately 40 PPI; or more particularly approximately 50 PPI. A distal region of inner braid (624) may have a braid density that linearly transitions from approximately 50 PPI to approximately 100 PPI; or more particularly from approximately 60 PPI to approximately 80 PPI, with the higher braid density being at the distal end. The braid density of inner braid (624) may linearly transition from the PPI value in the proximal region to the PPI values in the distal region. In some versions, outer braid (660) has a braid density ranging from approximately 40 picks per inch (PPI) to approximately 120 PPI; or more particularly approximately 80 PPI. In some such versions, the braid density of outer braid (660) is constant along the length of outer braid (660). In some other versions, the braid density of outer braid (660) varies along the length of outer braid (660). The braid density examples provided above are merely for illustrative purposes. Any other suitable braid densities may be used.

Body (602) is formed about the exterior of outer braid (660). By way of example only, body (602) may be formed about the exterior of outer braid (660) through a reflow process and/or through any other suitable process. At least some of the material used to form the region of body (602) outside of outer braid (660) may also reach the region between outer braid (660) and inner braid (624), as shown in FIG. 8. In scenarios where tendons (642) and/or stiffening wires (650) would otherwise tend to exert outwardly directed forces on body (502) (e.g., during bending of elongate member (600), particularly during driven articulation), outer braid (660) may effectively absorb such forces and thereby shield body (602) from any damage that might otherwise be caused to body (602) by tendons (642) and/or stiffening wires (650). In some versions, body (602) is softer in the distal region of elongate member (600) than in the proximal region of elongate member (600). Such differences in softness may provide greater stiffness in the proximal region than in the distal region. Such differences in softness may be achieved by providing body (602) with different thicknesses in the proximal and distal regions, by using different material and/or processes to form body (602), or by varying some other aspect of the configuration or method of manufacture of elongate member (600).

C. Example of Elongate Member with Coaxial Shafts, Coaxial Braided Structures, and Biplanar Articulation

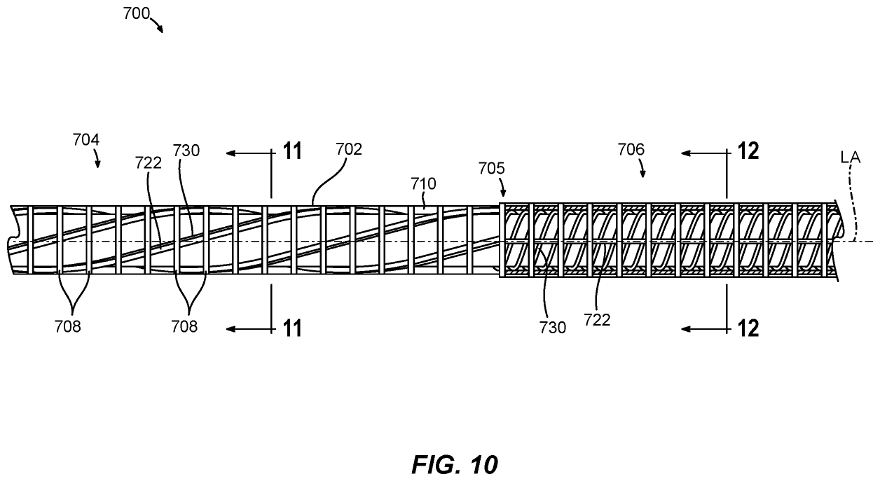

As noted above, it may be desirable to provide an elongate member that has a non-articulating proximal portion and an articulating distal portion, with tendons and stiffening wires having varied relative positioning to achieve such longitudinally dependent functionality of the elongate member. As also noted above, it may be desirable to incorporate braided structures into an elongate member to provide enhanced structural integrity without substantially compromising flexibility or cross-sectional efficiency. To these ends, FIGS. 10-12 show an example of an elongate member (700) representing a variation of elongate members (500, 600). Elongate member (700) may be constructed and operable like elongate members (500, 600) except as otherwise described below.

Elongate member (700) of the present example includes an outer body (702), with a proximal portion (704), a distal portion (706), and a transition (705) between proximal portion (704) and distal portion (706). In the present example, distal portion (706) is operable to articulate, such that the distal end (not shown) of elongate member (700) may be deflected laterally away from and toward a central longitudinal axis (LA) (e.g., defined by proximal portion (704)). Proximal portion (704) may be coupled with instrument coupling (11) of robotic surgical system (10), such that robotic surgical system (10) is operable to drive elongate member (700) via instrument coupling (11). The distal end of elongate member (700) may include any of the features and functionalities referenced above in the context of distal end (504) of elongate member (500).

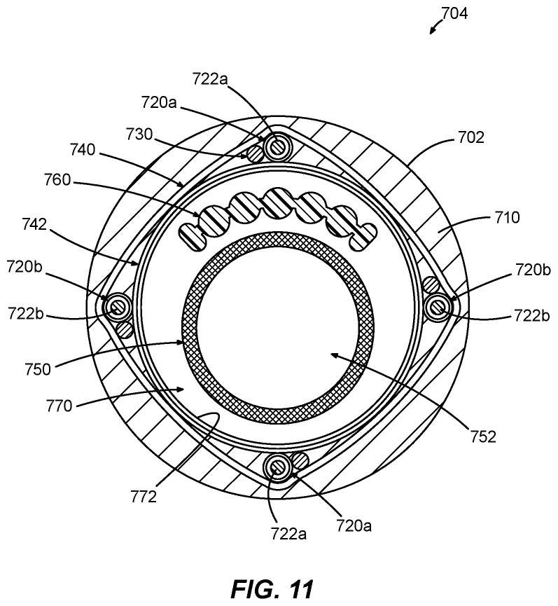

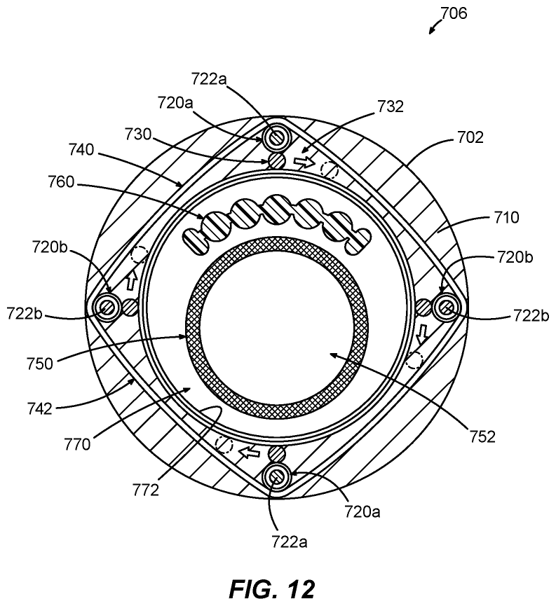

As shown in FIGS. 11-12, elongate member (700) includes an inner shaft (750) that defines a working channel (752). In some versions, inner shaft (750) is laterally offset relative to the central longitudinal axis (LA) of outer body (702). In some other versions, inner shaft (750) is coaxial with the central longitudinal axis (LA) of outer body (702). Working channel (752) is configured to slidably receive another instrument. By way of example only, basket (35) and basketing sheath (37) of basketing device (30) may be advanced distally via working channel (752). Like working channel (520) described above, working channel (752) may extend all the way to the distal end of elongate member (700), where working channel (752) may terminate in a distal opening allowing an instrument that is disposed in working channel (752) to exit distally from elongate member (700). In some versions, working channel (752) includes a liner (not shown) that is configured to reduce friction and thereby promote slidability through working channel (752). By way of example only, such a liner may include polytetrafluoroethylene (PTFE), polyimide, and/or any other suitable kind(s) of material(s).

A set of electrical wires (760) extend along a space (770) that is defined between inner shaft (750) and a liner (772). In some versions, electrical wires (760) are provided in the form of a ribbon cable. As another example, electrical wires (760) may be provided as traces on a flex circuit. Alternatively, electrical wires (760) may take any other suitable form. Electrical wires (760) may be configured to provide electrical communication to/from one or more components at the distal end of elongate member (500), including but not limited to one or more cameras, one or more light sources, one or more position sensors, one or more electrodes, etc. Electrical wires (760) may also be coupled with one or more corresponding components of robotic system (10), control system (50) and/or other devices. Liner (772) may include a low-friction material such as polytetrafluoroethylene (PTFE), polyimide, and/or any other suitable kind(s) of material(s). In some versions, the exterior of inner shaft (750) also include a low-friction material such as polytetrafluoroethylene (PTFE), polyimide, and/or any other suitable kind(s) of material(s). While not shown, some versions of elongate member (700) may provide electrical wires (760) along a helical path through space (770), such that electrical wires (760) define a helix along the length of elongate member (700), wrapped about the central longitudinal axis (LA).

An inner braid (742) is disposed outside of liner (772). Inner braid (742) may be constructed and operable just like inner braid (624) described above. A set of tendon lumens (720) and stiffening wires (730) are disposed outside of inner braid (742). Each tendon lumen (720) slidably contains a respective tendon (722). In some versions, each tendon lumen (720) is lined with a low-friction material such as polytetrafluoroethylene (PTFE), polyimide, and/or any other suitable kind(s) of material(s). Tendon lumens (720) and tendons (722) are provided in two diametrically opposing pairs in the present example. In particular, tendon lumens (720) and tendons (722) include a first pair of tendon lumens (720a) and corresponding tendons (722a) that are angularly spaced apart from each other by approximately 180 degrees about the central longitudinal axis (LA). Tendon lumens (720) and tendons (722) further include a second pair of tendon lumens (720b) and corresponding tendons (722b) that are angularly spaced apart from each other by approximately 180 degrees about the central longitudinal axis (LA). Tendon lumens (720a) and tendons (722a) of the first pair are angularly spaced apart from tendon lumens (720b) and tendons (722b) of the second pair by approximately 90 degrees about the central longitudinal axis (LA).

It should be understood from the teachings herein that tendons (722a) of the first pair are operable to drive articulation of distal portion (706) along a first plane of articulation; while tendons (722b) of the second pair are operable to drive articulation of distal portion (706) along a second plane of articulation, with the second plane of articulation being perpendicular to the first plane of articulation. Instrument coupling (11) may include features that are operable to drive a tendon (722a) of the first pair independently of a tendon (722b) of the second pair. Instrument coupling (11) may also drive tendons (722) of each pair simultaneously. Such driving may be accomplished by selectively pulling a selected tendon (722a) of the first pair and/or a selected tendon (722b) of the second pair.

Stiffening wires (730) are positioned approximately 180 degrees apart from each other about the central longitudinal axis (LA). The distal end of each stiffening wire (730) is fixed at the distal end of body (702); while the proximal end of each stiffening wire (730) is also fixed relative to shaft (730) such that stiffening wires (730) are not configured to translate longitudinally. Each stiffening wire (730) is formed of a non-compressible material having a high tensile strength. By way of example only, each stiffening wire (730) may comprise stainless steel, tungsten, hyten, LCP, aramid fiber, and/or any other suitable material(s). Stiffening wires (730) may be in the form of a monofilament, a braided structure, a bundle of filaments, or any other suitable form.

As shown in FIGS. 10-11, stiffening wires (730) are positioned in close angular proximity to corresponding tendons (722) along proximal portion (704) of elongate member (700), such that stiffening wires (730) effectively run alongside corresponding tendons (722), along proximal portion (704) of elongate member (700). As also shown in FIG. 10, stiffening wires (730) and tendons (722) extend along respective helical paths about the central longitudinal axis (LA) along proximal portion (704). By way of example only, such helical paths may have a pitch ranging from approximately 0.075 winds per inch to approximately 1.000 winds per inch; or more particularly may be approximately 0.200 winds per inch. Alternatively, any other suitable pitch may be provided. Upon reaching transition (705), as also shown in FIG. 10, tendons (722) transition from their respective helical paths to respective straight paths that run parallel with the central longitudinal axis (LA). Tendons (722) continue along these straight paths through distal portion (706).

Stiffening wires (730) continue along respective helical paths through distal portion (706), but the respective helical paths of stiffening wires (730) have a substantially higher density along distal portion (706), such a change in pitch occurs at transition (705); and is represented by arrow (732) in FIG. 12. By way of example only, the helical paths of stiffening wires (730) along distal portion (706) may have a pitch ranging from approximately 0.075 winds per inch to approximately 100 winds per inch. Alternatively, any other suitable pitch may be provided. In versions where electrical wires are positioned adjacent to stiffening wires (730) within body (702), such electrical wires may follow alongside stiffening wires (730) and thereby encounter the same changes in pitch as stiffening wires (730). Such an arrangement may prevent cyclic elongation and compression of the electrical wires during articulation.

Despite having a substantially denser helical path of stiffening wires (730) in distal portion (706) in the present example, the functionality of stiffening wires (730) in elongate member (700) may be substantially similar to the functionality of stiffening wires (550) in elongate member (500) as described above. In proximal portion (704), stiffening wires (730) may effectively function like a stiff spring; while in distal portion (706), stiffening wires (730) may effectively function like a soft spring. This differential in stiffness may ultimately focus the articulation to distal portion (706). In other words, the relatively high pitch of the helix formed by stiffening wires (730) along distal portion (706) may accommodate substantial flexibility along distal portion (706), such that stiffening wires (730) will not substantially impede articulation along distal portion (706). In some variations, the pitch of stiffening wires (730) along distal portion (706) may be varied to provide variable stiffness along distal portion (706), to thereby alter the bend radius or create preferential bending in different regions of distal portion (706).

An outer braid (740) is radially positioned outside of tendon lumens (720) and stiffening wires (730) in the present example. Lines (708) in FIG. 10 are intended to schematically represent outer braid (740), though it should be understood that outer braid (740) may in fact extend continuously along the lengths of proximal and distal portions (704, 706). While inner braid (742) has a substantially circular cross-sectional profile as shown in FIGS. 11-12, outer braid (740) does not have a substantially circular cross-sectional profile in this example. This is due to the process of manufacturing elongate member (700), where outer braid (740) is formed by wrapping the strands or wires of outer braid (740) about the exterior of inner braid (742), tendon lumens (720), and stiffening wires (730). An example of such a process, and devices that may be used to perform such a process, are described in greater detail below.

Body (702) is formed about the exterior of outer braid (740). By way of example only, body (702) may be formed about the exterior of outer braid (740) through a reflow process and/or through any other suitable process. At least some of the material used to form the region of body (702) outside of outer braid (740) may also reach the region between outer braid (740) and inner braid (742), as shown in FIGS. 11-12. In scenarios where tendons (722) and/or stiffening wires (730) would otherwise tend to exert out wardly directed forces on body (702) (e.g., during bending of elongate member (700), particularly during driven articulation), outer braid (740) may effectively absorb such forces and thereby shield body (702) from any damage that might otherwise be caused to body (702) by tendons (722) and/or stiffening wires (730). In some versions, body (702) is softer in the distal region of elongate member (700) than in the proximal region of elongate member (700). Such differences in softness may provide greater stiffness in the proximal region than in the distal region. Such differences in softness may be achieved by providing body (702) with different thicknesses in the proximal and distal regions, by using different material and/or processes to form body (702), or by varying some other aspect of the configuration or method of manufacture of elongate member (700).

D. Example of Apparatus for Manufacturing Elongate Member with Braided Structures

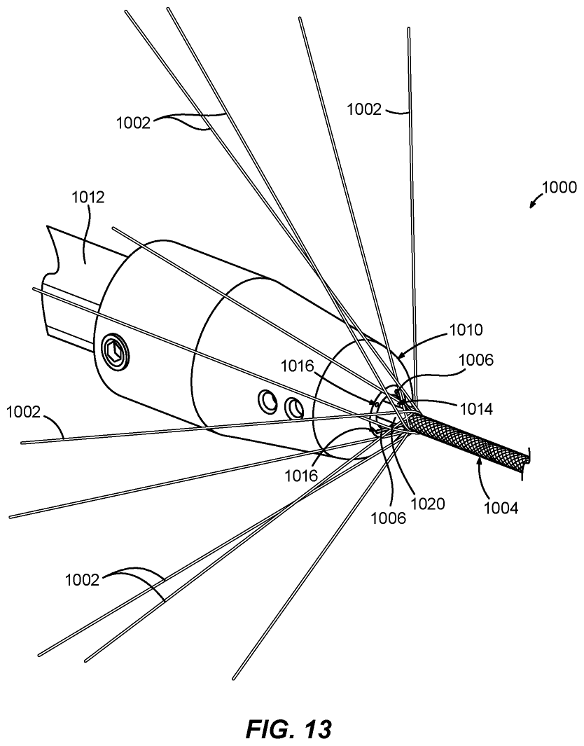

FIG. 13 depicts an example of an apparatus (1000) that may be used in a process to manufacture at least part of an elongate member that includes braided structures. In particular, FIG. 13 depicts a head (1010) at the distal end of a shaft (1012), with a mandrel (1020) disposed in a central opening (1014) defined by head (1010). Head (1010) has a rounded, generally conical shape in this example. The proximal end of shaft (1012) may be coupled with a motor (not shown), which may be operable to rotate shaft (1012) and head (1010) about the central longitudinal axis shared by shaft (1012) and mandrel (1020). An actuator (not shown) may be coupled with mandrel (1020) to drive mandrel (1020) longitudinally relative to shaft (1012). Alternatively, an actuator may be coupled with shaft (1012) to drive shaft (1012) and head (1010) longitudinally relative to mandrel (1020).

In addition to having central opening (1014), head (1010) includes an array of distal perimeter openings (1016) angularly spaced apart from each other about central opening (1014). Each distal perimeter opening (1016) is configured to slidably receive a corresponding inner tubular member or strand (1006). By way of example only, strand (1006) may take the form of an electrical wire lumen (530, 630) with a liner and an electrical wire (532, 632) therein, a tendon lumen (540, 640, 720) with a liner and a tendon (542, 642, 722) therein, a stiffening wire (550, 650, 730), and/or any other suitable structure(s). Head (1010) is configured to guide strands (1006) onto mandrel (1020) via distal perimeter openings (1016) as mandrel (1020) is translated longitudinally relative to head (1010) (or as head (1010) is translated longitudinally relative to mandrel (1020)). In some versions, a liner (622, 772), inner braid (624, 742), and/or other structure(s) is/are predisposed on mandrel (1020), such that strands (1006) are guided directly onto such predisposed structure(s). In some cases, head (1010) is rotated relative to mandrel (1020) as mandrel (1020) is translated longitudinally relative to head (1010) (or as head (1010) is translated longitudinally relative to mandrel (1020)). In such scenarios, strands (1006) may be applied to mandrel (1020) (or onto structure(s) predisposed on mandrel (1020)) in a helical configuration.

Regardless of whether head (1010) rotates relative to mandrel (1020) during longitudinal translation of mandrel (1020) relative to head (1010) (or during longitudinal translation of head (1010) relative to mandrel (1020)), a plurality of braid strands (1002) may be wrapped about mandrel (1020) (or onto structure(s) predisposed on mandrel (1020)) and about strands (1006) to thereby form a braid about strands (1006) (and about any structure(s) predisposed on mandrel (1020)). Such braid-wrapping of strands (1002) may be carried out through a coordinated movement of various spools, etc., from which strands (1002) are fed, using known components and techniques. The rounded, generally conical shape of head (1010) may support strands (1002) and thereby assist in guiding strands (1002) into place along mandrel (1020) (or onto structure(s) predisposed on mandrel (1020)) as strands (1002) are being wrapped to form the braid.

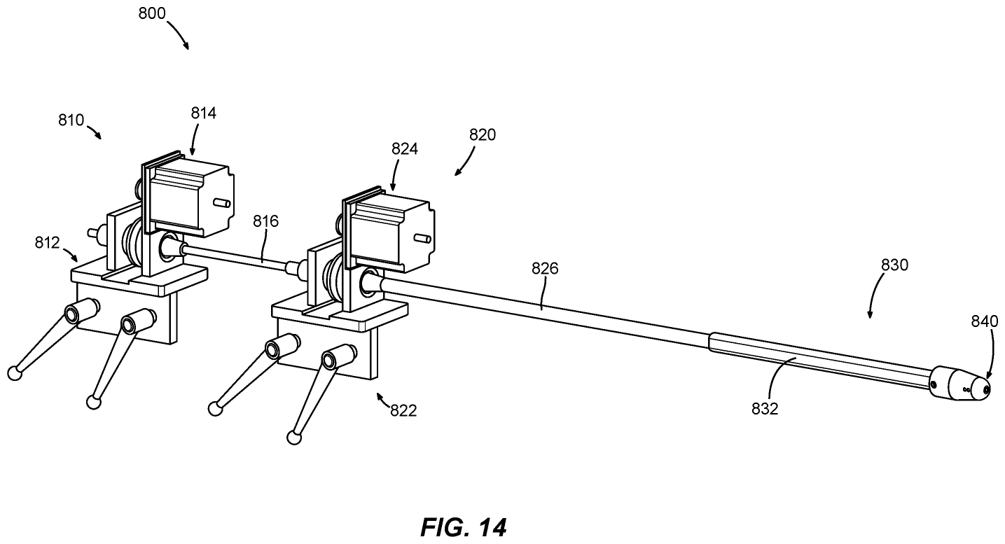

To the extent that apparatus (1000) is operable to form a braid around a single set of strands (1006) and about any structure(s) predisposed on mandrel (1020), it may be desirable to provide further complexity to the final structure, such as by providing variation in the arrangements of different strands (1006). To that end, FIG. 14 shows an alternative apparatus (800) that may be used to form structures more complex than those formed by apparatus (1000). Except as otherwise described below, apparatus (800) may be configured and operable similar to apparatus (1000). Apparatus (800) of this example includes a first subassembly (810) and a second subassembly (820). First subassembly (810) includes a base (812), a motor (814), and a shaft (816). First subassembly (810) is configured to be fixedly secured relative to a table or other fixed structure. Motor (814) is operable to rotate shaft (816) about the longitudinal axis defined by shaft (816). Second subassembly (820) includes a base (822), a motor (824), and a shaft (826). First subassembly (820) is configured to be fixedly secured relative to a table or other fixed structure. Motor (824) is operable to rotate shaft (826) about the longitudinal axis defined by shaft (826). By way of example only, each motor (814, 824) may include a stepper motor or any other suitable kind of motor.

Shaft (816) is coaxially disposed in shaft (826). Shaft (816) is also rotatable relative to shaft (826). With motors (814, 824) being independent of each other in this example, shaft (816) may rotate independently of shaft (826); and vice versa. An outer tube (832) is positioned about the distal portion of shaft (826). In some versions, outer tube (832) is fixedly secured to shaft (826) such that outer tube (832) will rotate unitarily with shaft (830).

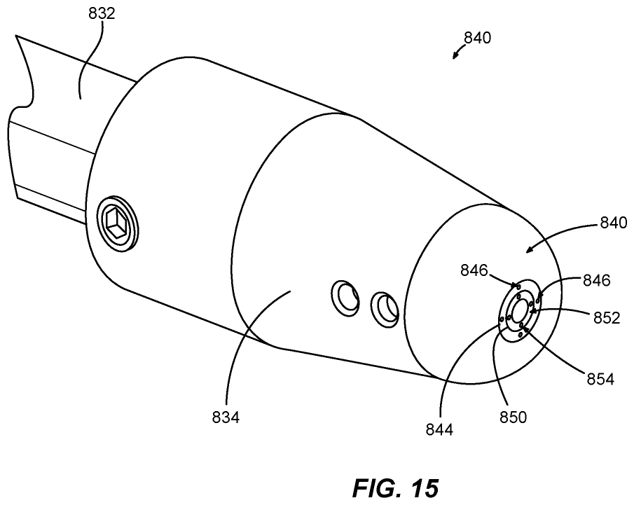

A head assembly (840) is located at the distal end of apparatus (800). As best seen in FIG. 15, head assembly (840) includes a frustoconical member (834), a rounded tip (842), a first annular feature (844), and a second annular feature (850). In the present example, frustoconical member (834) is unitarily integral with outer tube (832), rounded tip (842) and first annular feature (844) are unitarily integral with shaft (826), and second annular feature (850) is unitarily integral with shaft (816). Thus, first annular feature (844) rotates unitarily with shaft (826) and second annular feature (850) rotates unitarily with shaft (816). First annular feature (844) defines an array of distal perimeter openings (846) that are similar to distal perimeter openings (1016) described above. Second annular feature (850) defines a central opening (852) that is similar to central opening (1014) described above, such that central opening (852) may receive a mandrel like mandrel (1020). Second annular feature (850) also defines an array of distal perimeter openings (854) that are similar to distal perimeter openings (1016) described above. All distal perimeter openings (846, 854) are arranged about central opening (852), with distal perimeter openings (846) being positioned radially outwardly from distal perimeter openings (850).

One example of use of apparatus (800) includes use during a process of manufacturing elongate member (700). In such an example, liner (772) may be positioned about the mandrel and inner braid (742) may be formed about liner (772) through a coordinated movement of various spools, etc., from which braid strands are fed, as noted above. With liner (772) and inner braid (742) disposed on the mandrel, the loaded mandrel may be positioned within central opening (852), tendon lumens (720) and tendons (722) may be loaded through distal perimeter openings (846), and stiffening wires (730) may be loaded through distal perimeter openings (854). Alternatively, tendons (722) may be loaded through distal perimeter openings (854), and stiffening wires (730) may be loaded through distal perimeter openings (846). In either case, the next step may include securing distal portions of tendon lumens (720), tendons (722), and stiffening wires (730) relative to the inner braid (742) disposed on the mandrel; and securing portions of the braid forming strands to the inner braid (742) disposed on the mandrel. Then, the mandrel may be translated distally relative to subassemblies (810, 820) while the braid strand feeding spools are manipulated to form outer braid (740). This process may result in tendon lumens (720), tendons (722), and stiffening wires (730) being captured between inner braid (742) and the newly formed outer braid (740).

As the mandrel is translated distally relative to subassemblies (810, 820) while the braid strand feeding spools are manipulated to form outer braid (740), either or both of motors (814, 824) may be activated to selectively rotate corresponding annular features (844, 850). In the scenario where tendon lumens (720) and tendons (722) are loaded through distal perimeter openings (846), the activation of motor (824) and resulting rotation of first annular feature (844) will result in tendon lumens (720) and tendons (722) being applied to inner braid (742) in a helical pattern as outer braid (740) is being formed. Since motor (824) may be selectively activated, tendon lumens (720) and tendons (722) may be applied to inner braid (742) in a combination of a straight (i.e., parallel to the central longitudinal axis (LA)) orientation and a helical orientation; in helical orientations with pitches that vary along the length of inner braid (742); and/or in any other suitable pattern(s). Thus, motor (824) may be activated to drive shaft (826) at a first angular velocity, to thereby apply tendon lumens (720) and tendons (722) along proximal portion (704) in a relatively coarse pitch. At transition (705), motor (824) may be deactivated to cease rotation of drive shaft (826), thereby applying tendon lumens (720) and tendons (722) along distal portion (706) at an orientation that is parallel to the central longitudinal axis (LA).

Similarly, in the scenario where stiffening wires (730) are loaded through distal perimeter openings (854), the activation of motor (814) and resulting rotation of second annular feature (850) will result in stiffening wires (730) being applied to inner braid (742) in a helical pattern as outer braid (740) is being formed. Since motor (824) may be selectively activated, stiffening wires (730) may be applied to inner braid (742) in a combination of a straight (i.e., parallel to the central longitudinal axis (LA)) orientation and a helical orientation; in helical orientations with pitches that vary along the length of inner braid (742); and/or in any other suitable pattern(s). Thus, motor (814) may be activated to drive shaft (816) at the above-noted first angular velocity, to thereby apply stiffening wires (730) in the relatively coarse pitch alongside tendon lumens (720) and tendons (722) along proximal portion (704). In other words, shafts (816, 826) may rotate in the same direction and at the same angular velocity during formation of proximal portion (704). At transition (705), motor (814) may be activated to drive shaft (826) at a second (faster) angular velocity, to thereby apply stiffening wires (730) in the relatively fine pitch along distal portion (706).

Once outer braid (740) has been wrapped about tendon lumens (720), tendons (722), stiffening wires (730), inner braid (742), and liner (772) to form a suitable length of elongate member (700), the resulting structure may be removed from the mandrel, electrical wires (760) and inner shaft (750) may be inserted into liner (772), and body (702) may be formed (e.g., via overmolding, a reflow process, and/or any other suitable process. In some variations, body (702) is formed before the assembly is removed from the mandrel.

While the foregoing example describes the ability of motors (814, 826) to be activated at different speeds to vary the pitch of helical patterns formed by tendon lumens (720), tendons (722), and stiffening wires (730), it should be understood that the pitch of these helical patterns may also be varied by varying a rate of translation of the mandrel relative to subassemblies (810, 820). While the foregoing example describes the use of head assembly (840) to apply tendon lumens (720), tendons (722), and stiffening wires (730), it should be understood that head assemblies (840) may be used to apply various other kinds of elongate components during formation of a braid, including but not limited to electrical wires. While two subassemblies (810, 820) are provided in the present example, one or more additional subassemblies may be provided. Such additional subassemblies may be configured similar to subassembly (810), with a respective additional shaft coaxially disposed within shaft (816) and a respective annular feature coaxially disposed within annular feature (850), etc.

### III. EXAMPLES OF COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus comprising: (a) an elongate body having a proximal portion and a distal portion, the distal portion terminating at a distal end, the elongate body defining a central longitudinal axis; (b) a first tendon extending through the elongate body, the first tendon having a distal end that is fixedly secured relative to the distal end of the elongate body, the first tendon being operable to drive articulation of the distal portion of the elongate body relative to the proximal portion of the elongate body; (c) a second tendon extending through the elongate body, the second tendon having a distal end that is fixedly secured relative to the distal end of the elongate body, the second tendon being operable to drive articulation of the distal portion of the elongate body relative to the proximal portion of the elongate body; (d) a first stiffening member extending through the elongate body, the first stiffening member having a distal end that is fixedly secured relative to the distal end of the elongate body; and (e) a second stiffening member extending through the elongate body, the second stiffening member having a distal end that is fixedly secured relative to the distal end of the elongate body; the first tendon and the first stiffening member being positioned adjacent to each other along the proximal portion; the second tendon and the second stiffening member being positioned adjacent to each other along the proximal portion; the first tendon and the first stiffening member being angularly offset from each other along the distal portion; the second tendon and the second stiffening member being angularly offset from each other along the distal portion.

### Example 2

The apparatus of Example 1, the first tendon and the second tendon being angularly offset from each other by approximately 180 degrees about the central longitudinal axis along at least part of a length of the body; the first stiffening member and the second stiffening member being angularly offset from each other by approximately 180 degrees about the central longitudinal axis along at least part of a length of the body.

### Example 3

The apparatus of Example 2, the first tendon and the second tendon being angularly offset from each other by approximately 180 degrees about the central longitudinal axis along the entire length of the body; the first stiffening member and the second stiffening member being angularly offset from each other by approximately 180 degrees about the central longitudinal axis along the entire length of the body.

### Example 4

The apparatus of any of Examples 1 through 3, the first tendon and the first stiffening member together extending along a first helical path along the proximal portion; the second tendon and the second stiffening member together extending along a second helical path along the proximal portion.

### Example 5

The apparatus of Example 4, the first tendon extending along a first straight path along the distal portion, the first straight path being parallel with the central longitudinal axis; the second tendon extending along a second straight path along the distal portion, the second straight path being parallel with the central longitudinal axis.

### Example 6

The apparatus of any of Examples 4 through 5, the first stiffening member extending along a third helical path along the distal portion; the second stiffening member extending along a fourth helical path along the distal portion.

### Example 7

The apparatus of Example 6, the third helical path having a finer pitch than the first helical path; the fourth helical path having a finer pitch than the second helical path.

### Example 8

The apparatus of any of Examples 1 through 7, the body defining a working channel, the working channel being sized to slidably receive an instrument.

### Example 9

The apparatus of Example 8, the working channel being coaxially positioned about the central longitudinal axis.

### Example 10

The apparatus of any of Examples 8 through 9, further comprising a low friction liner in the working channel.

### Example 11

The apparatus of any of Examples 8 through 10, further comprising an inner braid the inner braid being positioned radially externally relative to the working channel, the inner braid being positioned radially internally relative to the first tendon, the second tendon, the first stiffening member, and the second stiffening member.

### Example 12

The apparatus of Example 11, the inner braid comprising metal wire strands.

### Example 13

The apparatus of any of Examples 1 through 12, further comprising an outer braid, the outer braid being positioned radially externally relative to the first tendon, the second tendon, the first stiffening member, and the second stiffening member, at least a portion of the body being positioned radially externally relative to the outer braid.

### Example 14

The apparatus of Example 12, the outer braid comprising metal wire strands.

### Example 15

The apparatus of any of Examples 1 through 14, further comprising: (a) a first tendon lumen having a low friction liner, the first tendon being slidably disposed in the first tendon lumen; and (b) a second tendon lumen having a low friction liner, the second tendon being slidably disposed in the first tendon lumen.

### Example 16

The apparatus of any of Examples 1 through 14, further comprising a plurality of electrical conduits extending along the elongate body.

### Example 17

The apparatus of Example 16, the electrical conduits comprising electrical wires.

### Example 18

The apparatus of Example 17, the electrical wires comprising a first electrical wire and a second electrical wire, the first electrical wire being angularly interposed between the first and second tendons along a first side of the central longitudinal axis, the second electrical wire being angularly interposed between the first and second tendons along a second side of the central longitudinal axis.

### Example 19

The apparatus of Example 18, the first stiffening member being positioned adjacent to the first electrical wire along the distal portion, the second stiffening member being positioned adjacent to the second electrical wire along the distal portion.

### Example 20

The apparatus of any of Examples 16 through 17, the electrical conduits being radially positioned inwardly relative to the first tendon and the first stiffening member.

### Example 21

The apparatus of Example 20, further comprising an inner shaft radially positioned inwardly relative to the electrical conduits.

### Example 22

An apparatus comprising: (a) an elongate body having a proximal portion and a distal portion, the distal portion terminating at a distal end, the elongate body defining a central longitudinal axis; (b) a first tendon extending through the elongate body, the first tendon having a distal end that is fixedly secured relative to the distal end of the elongate body, the first tendon being operable to drive articulation of the distal portion of the elongate body relative to the proximal portion of the elongate body; (c) a first stiffening member extending through the elongate body, the first stiffening member having a distal end that is fixedly secured relative to the distal end of the elongate body; (d) an inner braid positioned radially internally relative to the first tendon and relative to the first stiffening member; and (e) an outer braid positioned radially externally relative to the first tendon and relative to the first stiffening member.

### Example 23

The apparatus of Example 22, the first tendon and the first stiffening member being positioned adjacent to each other along the proximal portion; the first tendon and the first stiffening member being angularly offset from each other along the distal portion.

### Example 24

The apparatus of any of Examples 21 through 23, further comprising: (a) a second tendon extending through the elongate body, the second tendon having a distal end that is fixedly secured relative to the distal end of the elongate body, the second tendon being operable to drive articulation of the distal portion of the elongate body relative to the proximal portion of the elongate body; and (b) a second stiffening member extending through the elongate body, the second stiffening member having a distal end that is fixedly secured relative to the distal end of the elongate body.

### Example 25

The apparatus of Example 24, the second tendon and the second stiffening member being positioned adjacent to each other along the proximal portion; the second tendon and the second stiffening member being angularly offset from each other along the distal portion.

### Example 26

The apparatus of any of Examples 22 through 25, at least a portion of the elongate body being positioned radially externally relative to the outer braid.

### Example 27

The apparatus of any of Examples 22 through 26, the elongate body comprising a plastic material.

### Example 28

The apparatus of any of Examples 22 through 27, the first tendon comprising a pull wire.

### Example 29

The apparatus of any of Examples 22 through 28, the first tendon comprising a braid or bundle.

### Example 30

The apparatus of any of Examples 22 through 29, the first stiffening member comprising a wire.

### Example 31

The apparatus of any of Examples 22 through 30, the first stiffening member comprising a braid or bundle.

### Example 32

The apparatus of any of Examples 22 through 31, the first tendon and the first stiffening member extending together along a first helical path through the proximal portion.

### Example 33

The apparatus of Example 32, the first tendon and the first stiffening member extending together along different respective paths through the distal portion.

### Example 34

The apparatus of Example 33, the first tendon extending along a straight path through the distal portion, the straight path being parallel with the central longitudinal axis.

### Example 35

The apparatus of any of Examples 33 through 34, the first stiffening member extending along a second helical path through the distal portion.

### Example 36

The apparatus of Example 35, the second helical path having a finer pitch than the first helical path.

### Example 37

An apparatus, comprising: (a) a first subassembly, the first subassembly comprising: (i) a first shaft, the first shaft having a distal end, (ii) a first motor operable to rotate the first shaft, and (iii) a first annular member at the distal end of the first shaft, the first annular member having a first annular array of openings, each opening of the first annular array of openings being configured to slidably receive a corresponding strand; and (b) a second subassembly, the second subassembly comprising: (i) a second shaft, the second shaft having a distal end, the first shaft being coaxially disposed within the second shaft, (ii) a second motor operable to rotate the second shaft, and (iii) a second annular member at the distal end of the second shaft, the second annular member having a second annular array of openings, each opening of the second annular array of openings being configured to slidably receive a corresponding strand, the first annular member being positioned coaxially within the second annular member.

### Example 38

The apparatus of Example 37, the first and second motors being operable independently of each other.

### Example 39

The apparatus of any of Examples 37 through 38, the first annular member further defining a central opening.

### Example 40

The apparatus of Example 39, further comprising a mandrel disposed in the central opening.

### Example 41

The apparatus of Example 40, the mandrel being operable to translate relative to the first and second subassemblies.

### Example 42

The apparatus of any of Examples 37 through 41, further comprising a head assembly, the head assembly including the first and second annular members.

### Example 43

The apparatus of Example 42, the head assembly further including a frustoconical member proximal to the first and second annular members.

### Example 44

The apparatus of any of Examples 42 through 43, the head assembly further comprising a rounded tip adjacent to the second annular member.

### Example 45

A method comprising: (a) translating a mandrel relative to a first subassembly and relative to a second subassembly, the first subassembly including a first strand deposition feature, the second subassembly including a second strand deposition feature, the second strand deposition feature being operable independently of the first strand deposition feature; (b) depositing a first strand on the mandrel via the first strand deposition feature while the mandrel translates relative to the first subassembly and relative to the second subassembly; (c) depositing a second strand on the mandrel via the second strand deposition feature while the mandrel translates relative to the first subassembly and relative to the second subassembly; and (d) wrapping a plurality of external strands about the first and second strands and about the mandrel to form an outer braid about the first and second strands and about the mandrel while the mandrel translates relative to the first subassembly and relative to the second subassembly.

### Example 46

The method of Example 45, further comprising rotating the first strand deposition feature about the mandrel while depositing the first strand on the mandrel to thereby deposit the first strand on the mandrel in a helical orientation.

### Example 47

The method of Example 46, further comprising rotating the second strand deposition feature about the mandrel while depositing the second strand on the mandrel to thereby deposit the second strand on the mandrel in a helical orientation.

### Example 48

The method of Example 47, the first strand deposition feature being rotated simultaneously with the second strand deposition feature during at least part of the steps of depositing the first strand on the mandrel and depositing the second strand on the mandrel.

### Example 49

The method of Example 48, the first strand deposition feature being rotated at a first angular rate, the second strand deposition feature being rotated at a second angular rate.

### Example 50

The method of Example 49, the first angular rate being equal to the second annular rate during at least part of the steps of depositing the first strand on the mandrel and depositing the second strand on the mandrel.

### Example 51

The method of any of Examples 49 through 50, the first angular rate being unequal to the second annular rate during at least part of the steps of depositing the first strand on the mandrel and depositing the second strand on the mandrel.

### Example 52

The method of any of Examples 48 through 51, further comprising ceasing rotation of the first strand deposition feature while continuing rotation of the second strand deposition feature during part of the steps of depositing the first strand on the mandrel and depositing the second strand on the mandrel.

### Example 53

The method of any of Examples 45 through 52, the mandrel having an inner structure positioned thereon, such that the first and second strands are deposited on the mandrel via the inner structure.

### Example 54

The method of Example 53, the inner structure including an inner braid.

### Example 55

The method of any of Examples 53 through 54, the inner structure including a low friction liner.

### IV. MISCELLANEOUS

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions described above may be designed to be disposed of after a single use, or they may be designed to be used multiple times. Versions may, in either or both cases, be reconditioned for reuse after at least one use. Reconditioning may include any combination of the steps of disassembly of the systems, instruments, and/or portions thereof, followed by cleaning or replacement of particular pieces, and subsequent reassembly. In particular, some versions of the systems, instruments, and/or portions thereof may be disassembled, and any number of the particular pieces or parts of the systems, instruments, and/or portions thereof may be selectively replaced or removed in any combination. Upon cleaning and/or replacement of particular parts, some versions of the systems, instruments, and/or portions thereof may be reassembled for subsequent use either at a reconditioning facility, or by an operator immediately prior to a procedure. Those skilled in the art will appreciate that reconditioning of systems, instruments, and/or portions thereof may utilize a variety of techniques for disassembly, cleaning/replacement, and reassembly. Use of such techniques, and the resulting reconditioned systems, instruments, and/or portions thereof, are all within the scope of the present application.

By way of example only, versions described herein may be sterilized before and/or after a procedure. In one sterilization technique, the systems, instruments, and/or portions thereof is placed in a closed and sealed container, such as a plastic or TYVEK bag. The container and system, instrument, and/or portion thereof may then be placed in a field of radiation that may penetrate the container, such as gamma radiation, x-rays, or high-energy electrons. The radiation may kill bacteria on the system, instrument, and/or portion thereof and in the container. The sterilized systems, instruments, and/or portions thereof may then be stored in the sterile container for later use. Systems, instruments, and/or portions thereof may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) an elongate body having a proximal portion and a distal portion, the distal portion terminating at a distal end, the elongate body defining a central longitudinal axis;
(b) a first tendon extending through the elongate body, the first tendon having a distal end that is fixedly secured relative to the distal end of the elongate body, the first tendon being operable to drive articulation of the distal portion of the elongate body relative to the proximal portion of the elongate body;
(c) a second tendon extending through the elongate body, the second tendon having a distal end that is fixedly secured relative to the distal end of the elongate body, the second tendon being operable to drive articulation of the distal portion of the elongate body relative to the proximal portion of the elongate body;
(d) a first stiffening member extending through the elongate body, the first stiffening member having a distal end that is fixedly secured relative to the distal end of the elongate body; and
(e) a second stiffening member extending through the elongate body, the second stiffening member having a distal end that is fixedly secured relative to the distal end of the elongate body;
the first tendon and the first stiffening member being positioned adjacent to each other along the proximal portion;
the second tendon and the second stiffening member being positioned adjacent to each other along the proximal portion;
the first tendon and the first stiffening member being angularly offset from each other along the distal portion;
the second tendon and the second stiffening member being angularly offset from each other along the distal portion.

2. The apparatus of claim 1, the first tendon and the second tendon being angularly offset from each other by approximately 180 degrees about the central longitudinal axis along at least part of a length of the body;
the first stiffening member and the second stiffening member being angularly offset from each other by approximately 180 degrees about the central longitudinal axis along at least part of a length of the body.

3. The apparatus of claim 2, the first tendon and the second tendon being angularly offset from each other by approximately 180 degrees about the central longitudinal axis along the entire length of the body;
the first stiffening member and the second stiffening member being angularly offset from each other by approximately 180 degrees about the central longitudinal axis along the entire length of the body.

4. The apparatus of claim 1, the first tendon and the first stiffening member together extending along a first helical path along the proximal portion;
the second tendon and the second stiffening member together extending along a second helical path along the proximal portion.

5. The apparatus of claim 4, the first tendon extending along a first straight path along the distal portion, the first straight path being parallel with the central longitudinal axis;
the second tendon extending along a second straight path along the distal portion, the second straight path being parallel with the central longitudinal axis.

6. The apparatus of claim 4, the first stiffening member extending along a third helical path along the distal portion;
the second stiffening member extending along a fourth helical path along the distal portion.

7. The apparatus of claim 6, the third helical path having a finer pitch than the first helical path;
the fourth helical path having a finer pitch than the second helical path.

8. The apparatus of claim 1, the body defining a working channel, the working channel being sized to slidably receive an instrument.

9. The apparatus of claim 8, the working channel being coaxially positioned about the central longitudinal axis.

10. The apparatus of claim 8, further comprising a low friction liner in the working channel.

11. The apparatus of claim 8, further comprising an inner braid the inner braid being positioned radially externally relative to the working channel, the inner braid being positioned radially internally relative to the first tendon, the second tendon, the first stiffening member, and the second stiffening member.

12. The apparatus of claim 11, the inner braid comprising metal wire strands.

13. The apparatus of claim 1, further comprising an outer braid, the outer braid being positioned radially externally relative to the first tendon, the second tendon, the first stiffening member, and the second stiffening member, at least a portion of the body being positioned radially externally relative to the outer braid.

14. The apparatus of claim 12, the outer braid comprising metal wire strands.

15. The apparatus of claim 1, further comprising:
(a) a first tendon lumen having a low friction liner, the first tendon being slidably disposed in the first tendon lumen; and
(b) a second tendon lumen having a low friction liner, the second tendon being slidably disposed in the first tendon lumen.

16. The apparatus of claim 1, further comprising a plurality of electrical conduits extending along the elongate body.

17. An apparatus comprising:
(a) an elongate body having a proximal portion and a distal portion, the distal portion terminating at a distal end, the elongate body defining a central longitudinal axis;
(b) a first tendon extending through the elongate body, the first tendon having a distal end that is fixedly secured relative to the distal end of the elongate body, the first tendon being operable to drive articulation of the distal portion of the elongate body relative to the proximal portion of the elongate body;
(c) a first stiffening member extending through the elongate body, the first stiffening member having a distal end that is fixedly secured relative to the distal end of the elongate body;
(d) an inner braid positioned radially internally relative to the first tendon and relative to the first stiffening member; and
(e) an outer braid positioned radially externally relative to the first tendon and relative to the first stiffening member.

18. The apparatus of claim 17, the first tendon and the first stiffening member being positioned adjacent to each other along the proximal portion;
the first tendon and the first stiffening member being angularly offset from each other along the distal portion.

19. The apparatus of claim 17, further comprising:
(a) a second tendon extending through the elongate body, the second tendon having a distal end that is fixedly secured relative to the distal end of the elongate body, the second tendon being operable to drive articulation of the distal portion of the elongate body relative to the proximal portion of the elongate body; and
(b) a second stiffening member extending through the elongate body, the second stiffening member having a distal end that is fixedly secured relative to the distal end of the elongate body.

20. A method comprising:
(a) translating a mandrel relative to a first subassembly and relative to a second subassembly, the first subassembly including a first strand deposition feature, the second subassembly including a second strand deposition feature, the second strand deposition feature being operable independently of the first strand deposition feature;
(b) depositing a first strand on the mandrel via the first strand deposition feature while the mandrel translates relative to the first subassembly and relative to the second subassembly;
(c) depositing a second strand on the mandrel via the second strand deposition feature while the mandrel translates relative to the first subassembly and relative to the second subassembly; and
(d) wrapping a plurality of external strands about the first and second strands and about the mandrel to form an outer braid about the first and second strands and about the mandrel while the mandrel translates relative to the first subassembly and relative to the second subassembly.
